(19) United States
(12) Patent Application Publication
(10) Pub. No.: US 2017/0023522 A1
(43) Pub. Date: Jan. 26, 2017

(54) SAMPLE SEPARATOR AND SAMPLE SEPARATION/ADSORPTION DEVICE

(71) Applicant: Sharp Kabushiki Kaisha, Sakai City, Osaka (JP)

(72) Inventors: Uichi MIDORIKAWA, Sakai City (JP); Tsuyoshi TANAKA, Sakai City (JP); Hiroshi OHKI, Sakai City (JP); Shinichi GOTO, Sakai City (JP)

(21) Appl. No.: 15/303,550

(22) PCT Filed: Dec. 4, 2015
(86) PCT No.: PCT/JP2015/084170

§ 371 Date: Oct. 12, 2016

(30) Foreign Application Priority Data
Dec. 4, 2014 (JP) 2014-245857

(51) Int. Cl. G01N 27/447; G01N 33/68

(52) U.S. Cl. CPC G01N 27/44739; G01N 27/44747; G01N 27/44743; G01N 33/6803

(57) ABSTRACT

A sample separator (1) comprising an accommodation part (10) that accommodates a separation medium for electrophoresis, and a support (20) formed from a porous material. The accommodation part (10) includes an internal space (10c) filled with the separation medium, and is provided with a supply port (10a) and discharge port (10b) that are in communication with the internal space (10c). The support (20) is provided so as to block the discharge port (10b).

### TECHNICAL FIELD

The present invention relates to a sample separator and a sample separation/adsorption device. This application is based upon and claims the benefit of priority from the prior Japanese Patent Application No. 2014-245857, filed Dec. 4, 2014 in Japan, and the entire contents of which are incorporated herein by reference.

### BACKGROUND ART

In molecular biology and biology, analysis is performed that separates and detects biomolecules such as individual proteins and nucleic acids from a specimen (sample) in which a plurality of proteins, nucleic acids and the like are contained. As a technique for separating the individual biomolecules from samples, poly-acrylamide gel (PAGE) electrophoresis has been know. PAGE is a method that uses a gel (separation medium) containing polyacrylamide as a carrier molecule, and detects a difference in the molecular weights of biomolecules.

In addition, the respective biomolecules thus separated are retained by allowing to adsorb on resinous sheets called transfer membranes. This operation may be called “transfer”.

The transferred biomolecules are detected by antigen-antibody reaction using antibodies marked with fluorescent reagent or the like. The technique of detecting biomolecules using such antibodies has been known as Western blotting.

In recent years, technology for automating the aforementioned separation of samples and detection of the respective biomolecules thus separated has been proposed (for example, refer to Patent Documents 1 and 2).

The devices proposed in Patent Documents 1 and 2 both have a sample separation unit accommodating a gel for performing electrophoresis of samples, and a transfer membrane (sample adsorption member, adsorbing member) that performs transfer of the biomolecules thus separated. With these devices, after performing separation of sample with the gel inside of the sample separation unit, a voltage is applied so as to extend over the sample separation unit and transfer membrane, in a state bringing the sample separation unit and transfer membrane into contact. By configuring in this way, the biomolecules separated in the sample separation unit can be further subjected to electrophoresis up to the transfer membrane, and it is possible to continuously perform the separation of sample and the transfer of the biomolecules thus separated (adsorption to transfer membrane) with one device.

Patent Document 1: Japanese Unexamined Patent Application, Publication No. 2007-292616

Patent Document 2: Japanese Unexamined Patent Application, Publication No. 2010-8376

### DISCLOSURE OF THE INVENTION

### Problems to be Solved by the Invention

With the technology described in the above-mentioned patent documents, when the contact area of the location at which the sample separation unit and transfer membrane contact fluctuates, the state of transfer will not be stable, and stable detection of biomolecules becomes difficult. For this reason, an apparatus that enables stable transfer has been demanded.

The present invention is a sample separator that enables to make samples in which a plurality of proteins or nucleic acids are contained to be favorably separated, as well as transfer stably to a transfer membrane. In addition, it provides a sample separation/adsorption device having such a sample separator that enables the stable continuous conduction of the separation of sample and adsorption to a transfer membrane.

### Means for Solving the Problems

One of aspect of the present invention provides a sample separator including: an accommodation part capable of accommodating a separation medium for electrophoresis; and a support body with a porous material as a formation material thereof, in which the accommodation part has an internal space capable of being filled with the separation medium, and is provided with a supply port and discharge port that are in communication with the internal space, and the support body is provided to block the discharge port.

In one aspect of the present invention, it may be configured to further include a retaining member that retains the support body at the discharge port.

In one aspect of the present invention, it may be configured to further include a separation medium accommodated in the internal space, in which the separation medium is formed integrally with the support body.

In one aspect of the present invention, the separation medium may be configured to be a polyacrylamide gel.

One aspect of the present invention provides a sample separation/adsorption device including: a first buffer solution tank that stores a first buffer solution; a second buffer solution tank that stores a second buffer solution; a first electrode disposed in the first buffer solution tank; a second electrode disposed in the second buffer solution tank; the above-mentioned sample separator that is disposed in the first buffer solution tank; and a transcriptional body that transfers a sample, in which the sample separator has the supply port that opens inside of the first buffer solution tank, and the discharge port positioned inside of the second buffer solution tank, the support body possessed by the sample separator contacts one face of the transcriptional body, and the second electrode contacts another face of the transcriptional body.

In one aspect of the present invention, the transcriptional body may be configured to be provided to be movable relative to the support body.

### Effects of the Invention

According to the present invention, it is possible to provide a sample separator that enables to make samples in which a plurality of proteins or nucleic acids are contained to be favorably separated, as well as transfer stably to a transfer membrane. In addition, it is possible to provide a sample separation/adsorption device having such a sample separator that enables the stable continuous conduction of the separation of sample and adsorption to a transfer membrane.

### BRIEF DESCRIPTION OF THE DRAWINGS

FIG. 1 is an illustrative drawing of a sample separator according to a first embodiment;

FIG. 2 is an illustrative drawing of a sample separator according to the first embodiment;

FIG. 3 is an illustrative drawing of a sample separator according to a second embodiment;

FIG. 4 is an illustrative drawing of a sample separator according to the second embodiment;

FIG. 5 is an outline perspective view of a sample separation/adsorption device according to a third embodiment;

FIG. 6 is a cross-sectional view of a sample separation/adsorption device according to the third embodiment; and

FIG. 7 is an illustrative drawing of a sample separation/adsorption device according to a fourth embodiment.

### PREFERRED MODE FOR CARRYING OUT THE INVENTION

### First Embodiment

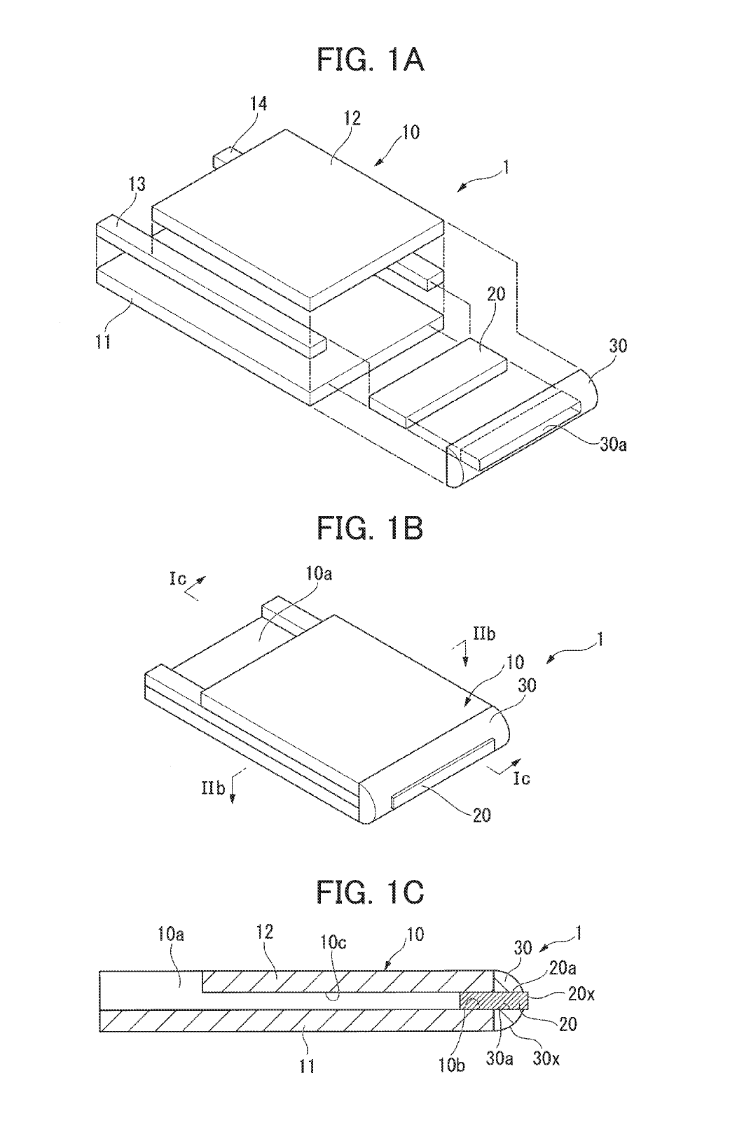

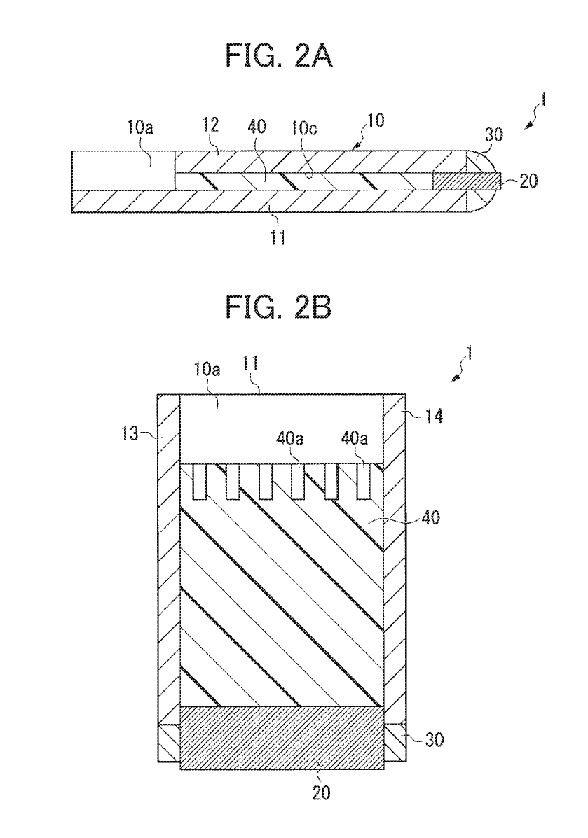

Hereinafter, a sample separator according to a first embodiment of the present invention will be explained while referencing FIGS. 1 and 2. It should be noted that, in order to easily view the drawings in all of the below drawings, the dimensions, ratios, etc. of each constituent element is varied as appropriate.

The sample separator according to the present invention explained hereinafter, and a sample separation device described later can be suitably employed in operations for separating by way of electrophoresis, and detecting samples that are a mixture of biomolecules.

It should be noted that “biomolecule” in the present disclosure is a biologically relevant molecule participating in reactions in a living body, and may be any of molecules derived from nature, synthetic molecules, and complexes containing either one or both of molecules derived from nature and synthetic molecules.

The biomolecules that are the analysis targets are normally high molecular weight compounds, and are preferably high molecular weight compounds derived from living bodies or biologically relevant, and proteins, deoxyribonucleic acid (DNA), ribonucleic acid (RNA), peptide nucleic acids, sugars, and complexes formed by at least two of these types bonding or interacting can be exemplified. In the case of the biomolecules being derived from living bodies, it may be derived from any of animals, plants and microorganisms.

The biomolecules separated by way of electrophoresis, transferred to the transfer membrane and detected by antibodies (detected by Western blotting) are often proteins or complexes containing proteins. The sample separator or sample separation/adsorption device of the present invention is used with such proteins or complexes containing proteins (sample) as the target.

FIG. 1 is an illustrative drawing of the sample separator 1. In the following explanation, the sample separator 1 may be referred to simply as “separator 1”. FIG. 1(a) is an exploded perspective view of the separator 1, FIG. 1(b) is a perspective view of the separator 1, and FIG. 1(c) is a cross-sectional view along the arrow of the line segment Ic-Ic in FIG. 1(b).

As shown in FIG. 1, the separator 1 has an accommodation part 10, a support body 20, and a retaining member 30.

### (Accommodation Part)

As shown in FIG. 1(a), the accommodation part 10 has a first member 11, second member 12, and spacers 13 and 14. The first member 11 and second member 12 are rectangular plate-like members in a plan view. The first member 11 and second member 12 are configurations in which the lengths in the short direction match, and the length in the long direction is longer for the first member 11 than the second member 12.

The spacers 13 and 14 are square rods having the same length as the length in the long direction of the first member 11.

The accommodation part 10 having the first member 11, second member 12, and spacers 13 and 14 may be formed using the same formation materials, or may be formed using different formation materials. As the formation material of the accommodation part 10, resin materials such as acrylic resin and polycarbonate resin; inorganic materials such as glass and ceramics; etc. can be exemplified. Either one or both of the first member 11 and second member 12 may be formed using a material having optical transparency.

Furthermore, on the first member 11 and second member 12, surface treatment for suitably retaining the separation medium described later may be conducted on the faces opposing each other.

As shown in FIGS. 1(a) and (b), upon assembling the accommodation part 10, first, the end positions in the short direction of the first member 11 and second member 12 are made to match in a plan view, and the positions on one end in the long direction are made to match. Then, in a state arranging the spacers 13 and 14 along the long direction of the first member 11 at both ends in the short direction of the first member 11, the spacers 13 and 14 are fixed by sandwiching with the first member 11 and second member 12.

As shown in FIGS. 1(b) and (c), the accommodation part 10 is a flattened, rectangular cylindrical structure having an internal space 10c, and having a supply port 10a at one end side, and a discharge port 10b at the other end side. The separation medium for electrophoresis described later is accommodated in the internal space 10c.

The supply port 10a opens largely at the second member 12. Operation during the filling of separation medium and during the supply of sample by the operator becomes easy, and thus workability improves.

### (Support Body)

The support body 20 is a right-angled parallelepiped member with a porous material as the formation material. As shown in FIG. 1, in the separator 1, the support body 20 is provided by configuring so as to block the discharge port 10b of the accommodation part 10. In addition, the support body 20 is provided to project from the discharge port 10b of the accommodation part 10.

The support body 20 may be fixed at the discharge port 10b by adhesive, double-sided tape, or crimping or heat sealing. As the adhesive, a vinyl acetate resin-based adhesive, or a synthetic rubber-based adhesive can be used.

Although described later in detail, as the separation medium accommodated in the accommodation part 10, gel-like materials containing water, and a carrier molecule of high molecular weight that supports water is suitably used. The support body 20 has a function of supporting such a separation medium in the discharge port 10b.

For this reason, the support body 20 is preferably a porous material having hydrophilicity on the order of having adherence with the separation medium, without inhibiting the gelation of the separation medium. For example, it is possible to suitably use felt of fibrillated resin (non-woven processed article). As the formation material of the fibrillated resin, polyethylene terephthalate (PET), polyethylene (PE), and polypropylene (PP) can be exemplified.

The fibrillated resin that is the formation material of the support body 20 may use only one type thereof, or may be a mixture of two or more types thereof. In addition, the fibrillated resin that is the formation material may be arrived at by performing additional treatment such as hydrophilization treatment thereon. As the support body 20, a felt with fibrillated resin of PET resin as the formation material is preferred.

Such a support body 20, for example, preferably has rigidity of an order that does not substantially deformed even when 10 N of pressure is applied thereto, for example. When using the support body 20 having such rigidity, the support body 20 hardly deforms during the transfer operation described later, and thus stable operation is possible. It should be noted that, although the support body 20 is explained as a right-angled parallelepiped member in the above description, it is not limited thereto, and may be any shape so long as able to support, such as film form.

### (Retaining Member)

The retaining member 30 is provided at an end of the accommodation part 10 on the side of the discharge port 10b. The retaining member 30 has a through hole 30a that is in communication with the discharge port 10b of the accommodation part 10. The through hole 30a is provided so as to be the same diameter from a side facing the discharge port 10b until the opposite side to the discharge port 10b.

The retaining member 30 accommodates the support body 20 projecting from the discharge port 10b of the accommodation part 10 inside of the through hole 30a, and retains a lateral face 20a of the support body 20. The support part 20 accommodated in the through hole 30a is arranged so that a position of an end face 20x matches an end of the through hole 30a, or projects slightly from the end of the through hole 30a.

In addition, as shown in FIG. 1(c), the retaining member 30 has an end face 30x on an opposite side to a side of the accommodation part 10 that curves in a cross-section in the arrow direction along the line segment Ic-Ic. For this reason, the leading end on the side of the discharge port 10b of the separator 1 is the end face 20x of the support body 20, when viewing the separator 1 in a cross-section along the arrow direction of the line segment Ic-Ic.

The retaining member 30 can be formed using the same formation material as the accommodation part 10.

During assembly of the separator 1, first, the support body 20 may be inserted into the through hole 30a of the retaining member 30, and subsequently, the support body 20 may be inserted in the discharge port 10b of the assembled accommodation part 10. By configuring in this way, the assembly operation becomes easy.

FIG. 2 is an illustrative drawing for one example of the separator 1 accommodating the separation medium in the internal space 10c. FIG. 2(a) is a cross-sectional view corresponding to FIG. 1(c), and FIG. 2(b) is a cross-sectional view along the arrow of the line segment IIb-IIb in FIG. 1(b).

The separation medium 40 retains biomolecules during electrophoresis, and enables the biomolecules to move inside thereof during electrophoresis. Gel electrophoresis normally uses a gelatinous separation medium containing water and a carrier molecule of high molecular weight that supports water.

In gel electrophoresis, when a potential difference is produced between one end side and the other end side of the separation medium handling the sample, the proteins that are charged particles will move within the gel. Upon doing so, due to the “molecular sieve effect” of interrupting movement of proteins by the carrier molecules in the gel, the movement distance of proteins per unit time during voltage application will differ based on differences in the charge, molecular weight, shape, etc. of proteins. As a result thereof, it is possible to individually separate a plurality of proteins contained in the analysis target.

The separation medium 40 can use anything so long as normally used in the aforementioned such electrophoresis. The formation material of the separation medium 40 may be appropriately selected according to the volume of sample that is the separation target, or type of biomolecules assumed to be contained in the sample, and polyacrylamide gel, dimethylacrylamide gel, agarose gel, etc. can be exemplified.

Such a separation medium 40 is obtained by pouring in a precursor (monomer and crosslinker) of the separation medium 40 from the supply port 10a of the separator 1, and causing to gel by polymerizing the precursor in the internal space 10c.

More specifically, after installing a cover for liquid leak prevention at the outer side of the support body 20, the precursor of the separation medium 40 is poured in from the supply port 10a, and gelled by polymerizing the precursor. The cover functions also as a protective member of the end face 20x of the support body 20 when trying to remove prior to use.

It may be configured to provide a plurality of grooves 40a at an end of the separation medium 40, as shown in FIG. 2(b), by arranging a comb-shaped jig on the side of the supply port 10a in advance prior to pouring in the precursor, and then removing the jig after pouring in the precursor until the jig is immersed. It is possible to control the intervals of the plurality of grooves 40a according to the shape of the jig. For example, by providing the plurality of grooves 40a at equal intervals, and injecting sample into the grooves 40a, the interval between the samples on the separation medium 40 tends to be controlled to equal intervals.

When accommodating the separation medium 40 in the aforementioned way, upon pouring the precursor into the internal space 10c, the precursor impregnates not only the internal space 10c, but also the support body 20 with a porous material as the formation material. When polymerizing the precursor in such a state, the separation medium 40 is accommodated in the internal space 10c, as well as being filled into the internal space of the support body 20, which is a porous material. For this reason, the separation medium 40 comes to be formed by one member from the end of the supply port 10a until the end face 20x of the support body 20. In addition, the separation medium 40 and support body 20 may be formed integrally. For example, the separation medium 40 and support body 20 may be formed integrally by the separation medium 40 impregnating the support body 20 as described above, or the separation medium 40 and support body 20 may be formed integrally by way of adhesive.

Since the separation medium 40 is a gel-like substance having water and a carrier molecule of high molecular weight in the aforementioned way, there may be shrinking depending on the change in water content of the gel or temperature change. For example, in a case of the separation medium 40 not being integrally formed with the support body 20 by arranging the support body 20 at the discharge port 10b after accommodating the separation medium 40, since the separation medium 40 isotropically shrinks, there is a risk of shrinking inside of the internal space 10c, and the separation medium 40 retracting from the side of the discharge port 10b. In this case, the separation medium 40 will separate from the support body 20, and there is a risk of defects arising in the separation operation of the sample, and the state of transfer of the sample described later.

However, as mentioned above, when adopting a structure in which the separation medium 40 and support body 20 are formed integrally, in the case of the separation medium 40 shrinking, the separation medium 40 will shrink by being drawn to the side of the separation body 20. For this reason, the separation medium 40 can constantly keep the position of the end on the side of the discharge port 10b, without retracting from the side of the discharge port 10b.

According to the separator 1 of the above such configuration, it is possible to provide a sample separator capable of causing samples in which a plurality of proteins or nucleic acids are contained to favorably separate, as well as stably transfer to a transfer membrane.

It should be noted that, although the separator 1 has the retaining member 30, it may be configured so as to constitute the separator by the accommodation part 10 and support body 20, without using the retaining member 30.

In addition, the accommodation part 10 is said to be configured by the first member 11, second member 12, and spacers 13 and 14; however, it is not limited thereto. For example, the accommodation part may be configured using the second member 12 and a structure in which the first member 11 and spacers 13, 14 are configured in a single member.

### Second Embodiment

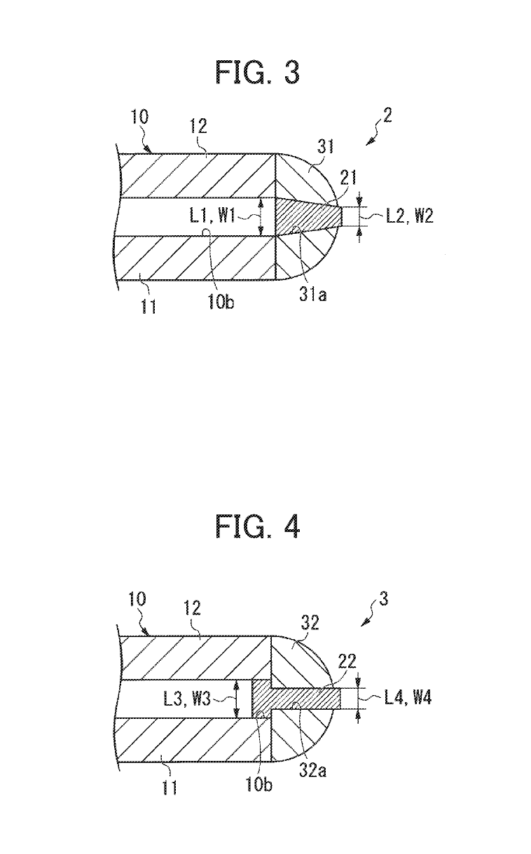

FIGS. 3 and 4 are illustrative drawings illustrating a sample separator according to a second embodiment of the present invention, and are partial cross-sectional views showing the shape of a leading-end side (discharge port side) of the sample separator. The sample separator according to the second embodiment differs from the sample separator 1 of the first embodiment in the shape of the leading-end side of the sample separator. For this reason, explanations will be omitted for members shared with the separator 1.

The sample separator 2 (separator 2) shown in FIG. 3 is provided with a retaining member 31 at an end of the accommodation part 10 on a side of the discharge port 10b thereof. The retaining member 31 has a through hole 31a that is in communication with the discharge port 10b of the accommodation part 10. The through hole 31a is provided so that an opening diameter L2 on an opposite side to the discharge port 10b is smaller than an opening diameter L1 on a side facing the discharge port 10b. Herein, a configuration in which the opening diameter L1 and opening diameter L2 vary is adopted by the width in the thickness direction (lamination direction of the first member 11 and second member 12) of the separator 2 varying.

The separator 2 has a support body 21. The support body 21 is a member with a porous material as the formation material, and the shape in a cross section in the viewing field of FIG. 3 is a trapezoidal shape in which the width in the thickness direction of the separator 2 gradually decreases. In other words, the support body 21 has a width L2 on the opposite side to the internal space that is smaller than the width W1 of the separator 2 on the internal space side thereof. It should be noted that, in the drawing, although the end of the support body 21 on the accommodation part 10 side thereof is positioned outside of the discharge port 10b of the accommodation part 10, at least a portion of the support body 21 may be accommodated in the discharge port 10b.

The sample separator 3 (separator 3) shown in FIG. 4 has a retaining member 32 provided at an end of the accommodation part 10 on a discharge port 10b side thereof. The retaining member 32 has a through hole 32a that is in communication with the discharge port 10b of the accommodation part 10. The through hole 32a is provided so as to have an opening diameter L4 that is smaller than the opening diameter L3 of the discharge port 10b.

The separator 3 has a support body 22. The support body 22 is a member with a porous material as the formation material. The support body 22 has an end on the discharge port 10b side thereof that overhangs in the thickness direction of the separator 3, in the shape of a cross section in the viewing field of FIG. 4. In other words, the support body 22 has a width L4 on the opposite side to the internal space that is smaller than the width W3 of the separator 3 on the internal space side thereof.

According to the separators 2, 3 of the above such configurations, it is possible to provide a sample separator for which it is possible to cause transfer to a transfer membrane stably, similarly to the aforementioned separator 1. In addition, by adopting the support bodies 21, 22, the dropping out of the support body is suppressed, and thus stable working becomes possible.

### Third Embodiment

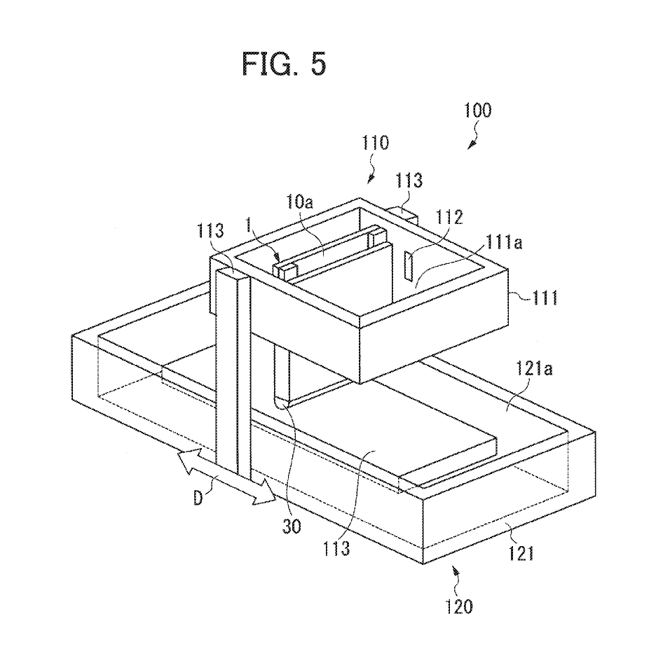

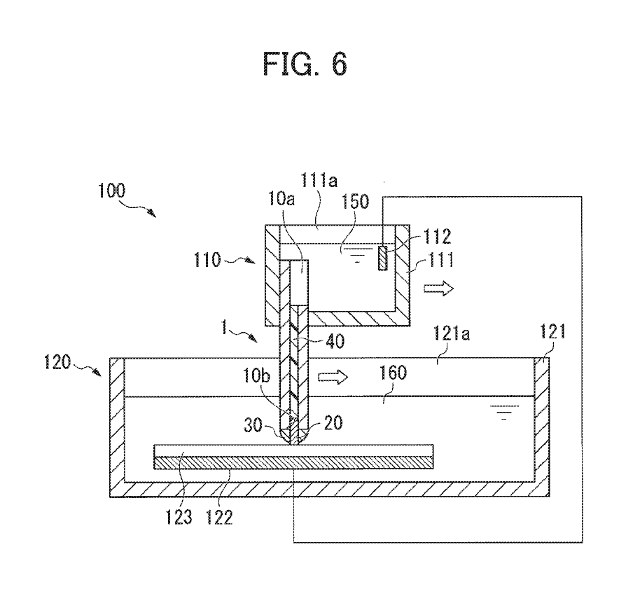

FIGS. 5 and 6 are illustrative drawings illustrating a sample separation/adsorption device 100 according to a third embodiment of the present invention. In the following explanation, the sample separation/adsorption device 100 may also be referred to simply as “separation/adsorption device 100”. FIG. 5 is an outline perspective view of the separation/adsorption device 100. FIG. 6 is a cross-sectional view of the separation/adsorption device 100, and is a view showing an appearance during use.

As shown in FIGS. 5 and 6, the separation/adsorption device 100 of the present embodiment has a sample separation unit 110, and sample adsorption unit 120.

The sample separation unit 110 includes a first buffer solution tank 111, first electrode 112, bridge part 113, and the aforementioned sample separator according to the present invention. FIG. 5 illustrates by having the separator 1 shown in the first embodiment as the sample separator.

The first buffer solution tank 111 is a container having an internal space 111a that stores the buffer solution. The separator 1 is installed in the first buffer solution tank 111.

The separator 1 installed in the first buffer solution tank 111 has a supply port 10a that opens at the internal space 111a of the first buffer solution tank 111. In addition, the leading end of the separator 1 at a side of the retaining member 30 is provided to project more downwards than the bottom of the first buffer solution tank 111.

The first electrode 112 is arranged at the internal space 111a of the first buffer solution tank 111. For example, it is possible to use a platinum electrode as the first electrode 112.

The bridge part 113 is a member that clamps the first buffer solution tank 111, and supports in a state lifted to a predetermined height direction. The bridge part 113 is provided to be movable in the thickness direction of the separator 1 (in the drawing, direction of white arrow indicated by symbol D), in a state supporting the first buffer solution tank 111. In addition, the bridge part 113 may be provided with an adjustment means for adjusting the height position of the first buffer solution tank 111.

The sample adsorption unit 120 includes a second buffer solution tank 121, second electrode 122 and transcriptional body 123.

The second buffer solution tank 121 is a container assuming a rectangle in a plan view, and having an internal space 121a that stores the buffer solution. The bridge part 113 of the sample separation unit 110 is arranged at both sides of the second buffer solution tank 121, and the first buffer solution tank 111 and separator 1 are arranged above the second buffer solution tank 121 by making to span the second buffer solution tank 121.

The separator 1 installed in the first buffer solution tank 111 has the discharge port 10b positioned at the internal space 121a of the second buffer solution tank 121.

The second electrode 122 is a plate-like member assuming a rectangle in a plan view, and is arranged in the internal space 121a of the second buffer solution tank 121. For example, it is possible to use a platinum electrode as the second electrode 122.

The second electrode 122 is electrically connected to the first electrode 122, thereby enabling a voltage to be applied between the first electrode 112 and the second electrode 122. At this time, the first electrode 112 is used as the cathode, and the second electrode 122 is used as the anode.

The transcriptional body 123 is a plate-like or film-like member assuming a rectangle in the plan view, and is a member that can adsorb and retain biomolecules. The formation material of the transcriptional body 123 may be appropriately selected according to the volume of sample that is the separation target, or the type of biomolecules that are assumed to be contained in the sample, and PVDF (polyvinylidene fluoride), nitrocellulose membrane, etc. can be exemplified.

The transcriptional body 123 is a layer on one side of the second electrode 122, and is arranged by orienting a face on an opposite side to the second electrode 122 side thereof towards a side of the sample separation unit 110.

As shown in FIG. 6, during use of the separation/adsorption device 100, a buffer solution 150 is stored in the first buffer solution tank 111, and a buffer solution 160 is stored in the second buffer solution tank 121. The buffer solution 150 is a buffer solution for the cathode, and the buffer solution 160 is a buffer solution for the anode.

The buffer solutions 150, 160 can employ buffer solutions of known compositions, and may be appropriately selected according to the volume of sample that is the separation target, or the type of biomolecules assumed to be contained in the sample. As preferred solutions, a tris(hydroxymethyl)aminomethane (Tris)/glycine buffer solution, Tris/glycine/sodium dodecyl sulfate (SDS) buffer solution, acetate buffer solution, sodium carbonate buffer solution, N-cyclohexyl-3-aminopropane sulfonic acid (CAPS) buffer solution, Tris/boric acid/ethylenediaminetetraacetic acid (EDTA) buffer solution, Tris/acetic acid/ethylenediaminetetraacetic acid (EDTA) buffer solution, 3-morpholinopropanesulfonic acid (MOPS) buffer solution, phosphate buffer solution, Tris/tricine buffer solution, Tris/methanol buffer solution, Tris/ethanol buffer solution, etc. can be exemplified.

During use of the separation/adsorption device 100, after supplying sample to the separation medium 40 from the supply port 10a of the separator 1, the separator 1 is set in the first buffer solution tank 111. At this time, the support body 20 of the separator 1 is pushed against the transcriptional body 123 with a predetermined pressure.

The “predetermined pressure” upon pushing the support body 20 against the transcriptional body 123 is preferably 0.1 N to 10 N, more preferably 2 N to 8 N, and even more preferably 5 N to 6 N. The upper limit value and lower limit value can be combined arbitrarily.

Subsequently, the buffer solution 150 is stored in the first buffer solution tank 111 and second buffer solution tank 121. In the first buffer solution tank 111, an amount of the buffer solution 150 reaching the supply port 10a is stored. The first electrode 112 is immersed in the buffer solution 150.

In addition, in the second buffer solution tank 121, an amount of the buffer solution 160 reaching the end face 20x of the support body 20, which is a leading end of the separator 1, is stored. The second electrode 122 and transcriptional body 123 are immersed in the buffer solution 160.

In such a state, a predetermined voltage is applied between the first electrode 112 and second electrode 122, thereby causing the biomolecules contained in the sample handled by the separation medium 40 of the separator 1 to undergo electrophoresis. The type of electrophoresis is not particularly limited, and may be appropriately selected according to the biomolecules subjected to electrophoresis. In the case of the biomolecules being proteins, employing an electrophoresis method such as SDS electrophoresis, Native electrophoresis, Blue Native electrophoresis, and two-dimensional electrophoresis can be exemplified.

The sample handled by the separation medium 40 of the separator 1 is separated based on differences in the charge, molecular weight, shape, etc. of the biomolecules on the separation medium 40, while moving within the separation medium 40 to the side of the support body 20 (separation operation).

Among the biomolecules contained in the sample, the bridge part 113 of the sample separation unit 110 starts movement along the edge of the second buffer solution tank 121, around the time when biomolecules having the fastest migration speed reach the support body 20. The support body 20 of the separator 1 and the transcriptional body 123 thereby move relatively.

In the transcriptional body 123, the position adsorbing biomolecules dispensed changes via the support body 20, accompanying movement of the support body 20. The transcriptional body 123 thereby retains the biomolecules separated by the separation medium 40 in a state separated without allowing to mix again (transfer operation).

After the transfer operation, it is possible to detect the types and amount of separated biomolecules, by removing the transcriptional body 123 on which the biomolecules are retained, and conducting a known method such as Western blotting.

According to the separation/adsorption device 100 of the above such configuration, since the aforementioned sample separator 1 of the present invention is used, it becomes possible to continuously conduct the separation of sample and adsorption to the transcriptional body stably.

### Fourth Embodiment

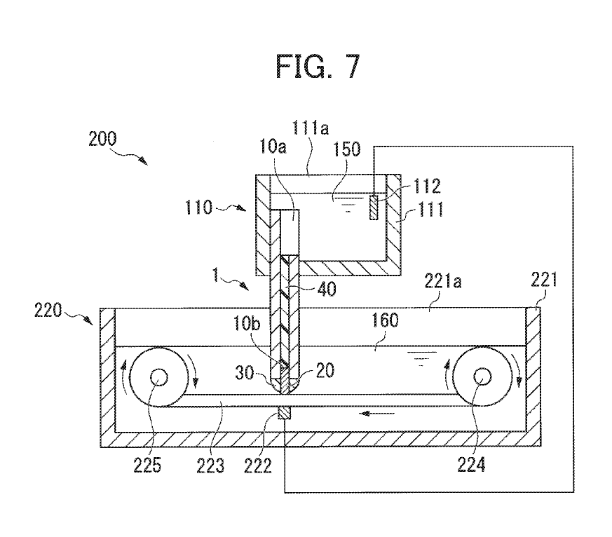

FIG. 7 is an illustrative drawing illustrating a sample separation/adsorption device 200 (separation/adsorption device 200) according to a fourth embodiment of the present invention, and is a view corresponding to FIG. 6.

The separation/adsorption device 200 has the aforementioned sample separation unit 110 and sample adsorption unit 220.

The sample adsorption unit 220 has a second buffer solution tank 221, second electrode 222, transcriptional body 223, unwinding roll 224, and winding roll 225.

The second buffer solution tank 221 can adopt a similar configuration as the aforementioned second buffer solution tank 121.

The second electrode 222 is a plate-like member that is rectangular in a plan view, having an identical size as the opening diameter of the discharge port 10b, for example, and is arranged below the support body 20 of the separator 1 at a position corresponding with the support body 20. For example, it is possible to use a platinum electrode as the second electrode 222.

The second electrode 222 is electrically connected to the first electrode 112, whereby it is possible to apply a voltage between the first electrode 112 and second electrode 222. At this time, the first electrode 112 is used as the cathode, and the second electrode 222 is used as the anode.

A transcriptional body 223 is a belt-like member extending in one direction, and is a member that can adsorb and retain biomolecules. As the formation material of the transcriptional body 223, it is possible to adopt the same material as the aforementioned transcriptional body 123.

The transcriptional body 223 is arranged at the unwinding roll 224 in a state wound up in a rolled form, and is unwound from the unwinding roll 224, passes between the support body 20 and second electrode 222, while being wound up at the winding roll 225.

During use of such a separation/adsorption device 200, after supplying the sample to the separation medium 40 from the supply port 10a of the separator 1, the separator 1 is set in the first buffer solution tank 111. At this time, the transcriptional body 223 is clamped between the support body 20 of the separator 1 and the second electrode 222, and the support body 20 is pushed against the transcriptional body 223 with a predetermined pressure.

The “predetermined pressure” upon pushing the support body 20 against the transcriptional body 223 is preferably 0.1 N to 10 N, more preferably 2 N to 8 N, and even more preferably 5 N to 6 N. The upper limit value and lower limit value can be combined arbitrarily.

In such a state, the buffer solution 150 is stored in the first buffer solution tank 111 and the second buffer solution tank 221. A predetermined voltage is applied between the first electrode 112 and second electrode 122, thereby causing the biomolecules contained in the sample handled by the separation medium 40 of the separator 1 to undergo electrophoresis.

Among the biomolecules contained in the sample, around the time when those having the fastest migration speed reach the support body 20, the transcriptional body 223 wound up in a rolled form from the unwinding roll 224 is unwound, and the winding roll 225 winds up. The support body 20 of the separator 1 and the transcriptional body 223 thereby move relatively.

In the transcriptional body 223, the position adsorbing biomolecules changes, accompanying movement of the transcriptional body 223. The transcriptional body 123 thereby retains the biomolecules separated by the separation medium 40 in a state separated without allowing to mix again (transfer operation).

After the transfer operation, it is possible to detect the types and amount of separated biomolecules, by removing the transcriptional body 223 on which the biomolecules are retained, and conducting a known method such as Western blotting.

Even if the separation/adsorption device 200 of the above such configuration, since the aforementioned sample separator 1 of the present invention is used, it becomes possible to continuously conduct the separation of sample and adsorption to the transcriptional body stably.

### EXAMPLES

Although the present invention will be explained by Examples hereinafter, the present invention is not to be limited to these examples.

It should be noted that a separator of the same configuration as the separator 1 explained in the first embodiment was used as the separator in the Examples. In addition, a device of the same configuration as the separation/adsorption device 100 explained in the third embodiment was used as the separation/adsorption device. For this reason, in the following explanation, an explanation is provided using the reference symbols attached in FIGS. 1 and 2 as appropriate.

### Example 1

The biomolecule samples employed a protein solution prepared by solubilizing biological tissue (mouse liver) by the respective aqueous solutions of 8 mol/L urea, 2 mol/L of thiourea, 4% by mass 3-(3-cholamidepropyl)dimethylammonio-1-propanesulphonate (CHAPS), and 20 mmol/L dithiothreitol (DTT), and extracting by removing insoluble impurities by way of centrifugal separation.

To the separator 1 to which the support body 20 formed with a porous material of polyethylene terephthalate is fixed, a 10% by mass acrylamide solution on which degassing was sufficiently performed under reduced pressure so that gel polymerization would not be inhibited was injected. To the acrylamide solution, tetramethyl ethylenediamide (TEMED) and ammonium persulfuric acid (APS) were added to cause gelation, thereby preparing the separation medium 40 of polyacrylamide gel.

In the first buffer solution tank 111, 180 mL of buffer solution for the cathode was stored, and 200 mL of buffer solution for the anode was stored in the second buffer solution tank 121.

As the buffer solution for the cathode, a mixed aqueous solution of 100 mmol/L of MOPS, 50 mmol/L of (bis(2-hydroxyethyl)amino-tris(hydroxymethyl) methane) (Bis-Tris), 50 mmol/L of Tris, and 0.25% SDS was used.

As the buffer solution for the anode, a mixed aqueous solution of 100 mmol/L of MOPS, 50 mmol/L of Bis-Tris, 50 mmol/L of Tris, and 20% by volume of EtOH was used.

After adding SDS solution to the above-mentioned protein solution and equilibrating, it was added to the separation medium 40 exposed at the supply port 10a side. Subsequently, separation of the biomolecules by way of SDS-polyacrylamide gel electrophoresis (SDS-PAGE) was performed, by applying a voltage at a constant current of 50 mA between the first electrode 112 and second electrode 122.

After adsorbing the biomolecules to the transcriptional body, blocking treatment was performed with 3% by mass skim milk, and it was confirmed that the band shapes of proteins could be detected by Western blotting.

### Example 2

First, using an electrophoresis device (Auto 2D, BM-100, manufactured by Sharp Corp.), the above-mentioned protein solution was separated with a unique electric charge to the IEF (isoelectric focusing) chip (one-dimensional electrophoresis). After impregnating the gel portion of the IEF chip with SDS solution and equilibrating, the gel portion of the IEF chip was set in the separation medium 40 exposed at the side of the supply port 10a.

Subsequently, similarly to Example 1, the separation of the biological sample was performed by way of SDS-polyacrylamide gel electrophoresis (SDS-PAGE) (two-dimensional electrophoresis).

Using the transfer membrane on which the proteins were retained, it was confirmed that specific proteins could be detected by way of Western blotting, similarly to Example 1.

In the present embodiment, due to spreading out the proteins by way of two-dimensional electrophoresis, the transfer membrane to which proteins were transferred in the present embodiment can be used as a protein chip having protein information such as molecular weight and isoelectric point.

According to the above results, it could be confirmed that the present invention is effective.

### INDUSTRIAL APPLICABILITY

The present invention is widely applicable in life science fields such as medicine, engineering, pharmacy, physics and agriculture.

### EXPLANATION OF REFERENCE NUMERALS

1, 2, 3 sample separator (separator)

20, 21, 22 support body

10 accommodation part

10a supply port

10b discharge port

10c internal space

30, 31, 32 retaining member

40 separation medium

100, 200 sample separation/adsorption device (separation/adsorption device)

111 first buffer solution tank

112 first electrode

121, 221 second buffer solution tank

122, 222 second electrode

123, 223 transcriptional body

150, 160 buffer solution

1. A sample separator, comprising:
an accommodation part capable of accommodating a separation medium for electrophoresis; and
a support body with a porous material as a formation material thereof,
wherein the accommodation part has an internal space capable of being filled with the separation medium, and is provided with a supply port and discharge port that are in communication with the internal space, and
wherein the support body is provided to block the discharge port.

2. The sample separator according to claim 1, wherein the support body projects from the discharge port.

3. The sample separator according to claim 1, further comprising a retaining member that retains the support body at the discharge port.

4. The sample separator according to claim 1, further comprising a separation medium filled into the internal space, wherein the separation medium is formed integrally with the support body.

5. The sample separator according to claim 1, wherein the separation medium is a polyacrylamide gel.

6. A sample separation/adsorption device, comprising:
a first buffer solution tank capable of storing a first buffer solution;
a second buffer solution tank capable of storing a second buffer solution;
a first electrode disposed in the first buffer solution tank;
a second electrode disposed in the second buffer solution tank;
a sample separator according to claim 1 that is disposed in the first buffer solution tank; and
a transcriptional body that transfers a sample,
wherein the sample separator has the supply port that opens inside of the first buffer solution tank, and the discharge port positioned inside of the second buffer solution tank,
wherein the support body possessed by the sample separator contacts one face of the transcriptional body, and
wherein the second electrode contacts another face of the transcriptional body.

7. The sample separation/adsorption device according to claim 6, wherein the transcriptional body is provided to be movable relative to the support body.
